(19) United States
(12) Patent Application Publication
(10) Pub. No.: US 2010/0089404 A1
(43) Pub. Date: Apr. 15, 2010

(54) Endotracheal and Tracheostomy Devices

(75) Inventors: Ronny Bracken, Monroe, GA (US); Silke Talsma, Atlanta, GA (US)

Correspondence Address: Rutan & Tucker, LLP., 611 ANTON BLVD, SUITE 1400, COSTA MESA, CA, 92626, US

(73) Assignee: C.R. Bard,Inc., Murray Hill (US)

(21) Appl. No.: 12/523,981

(22) PCT Filed: Jan. 24, 2008
(86) PCT No.: PCT/US08/51891

§ 371 Date: Dec. 7, 2009

Related U.S. Application Data
(60) Provisional application No. 60/886,574, filed on Jan. 25, 2007.

(51) Int. Cl. A61M 16/04; A61L 15/44; A61L 15/46

(52) U.S. Cl. 128/207.14; 604/265

(57) ABSTRACT

An endotracheal device may include an elongated body member having a first end and a second end, first and second lumen defined by the body member, and a funnel member proximal the second end. The first lumen may extend from a first opening in the first end to a first opening in the second end The second lumen may extend from a second opening in the first end to at least one point in a wall of the body member proximal the second end. The tunneling member may be configured to direct fluid toward at least one of the points in the wall.

This application claims benefit of priority to U.S. Provisional Patent Application No. 60/886,574, filed Jan. 25, 2007, entitled “Endotracheal and Tracheostomy Devices.”

The present disclosure is directed to medical devices. More particularly, the present disclosure is directed to endotracheal and tracheostomy devices.

Normal ventilation results from the contraction and expansion of the diaphragm, leading to the cyclic flow of oxygen into, and carbon dioxide out of the lungs. Mechanical ventilation may be indicated for critical care and surgical patients unable to ventilate on their own. Mechanical ventilation is generally provided with an endotracheal tube fluidly coupled to a mechanical ventilator. Morbidity and mortality resulting from both mechanical ventilation and the underlying condition can be significant. A major contributor to the morbidity and mortality is ventilator-assisted pneumonia (“VAP”). Colonization of the lower respiratory tract, especially with gram negative bacteria, may be an early stage in the development of VAP. Aspiration of colonized secretions is a leading cause of VAP.

During use of conventional endotracheal tubes, mucus often accumulates on the surface of the tubes. The mucus originates at the tracheal tissue, which is coated with layers of mucus secretions that play a major role in host defense. Intubated patients cannot clear their airways by expectoration, and some may suffer from mucus hypersecretion. To ensure proper ventilation, the airway must be cleared of excess mucus. Such removal may be effectuated mechanically, for example, by shaving, and frequent suctioning may be required. Mucus shaving and/or suctioning may be uncomfortable and/or painful to patients. In addition, clearance of airway secretions using suction catheters and other devices via the endotracheal tube can substantially increase the risk of nosocomial infections, especially VAP. Accordingly, it may be desirable to remove mucus from endotracheal tubes without mechanical intervention and in a more comfortable manner.

Conventional endotracheal tubes may be either flexible or relatively stiff. Some adult tubes have an inflatable member to seal the lower airways against air leakage and aspiration of secretions, while smaller pediatric tubes generally are unmembered.

With some conventional endotracheal tubes, attempts have been made improve suctioning efficiency by modifying suctioning orifice geometry, by modifying member design to create a collection basin, and by interchanging suction devices. It may be desirable to provide an endotracheal tube that simply and inexpensively directs fluids to the suctioned region.

A tracheotomy is a relatively common procedure for bypassing a tracheal obstruction. A tracheostomy tube is inserted into the trachea by making an incision in neck tissue. The tracheostomy tube provides access to the trachea below the larynx in the neck of a patient. The primary objective in using a tracheostomy tube is to provide adequate airway ventilation. The tube can permit passive ventilation, i.e., ventilation occurring without assistance. Alternatively, the tracheostomy tube can provide access for mechanically assisted ventilation for patients that are unable to breathe on their own. It is not unusual for the tracheostomy stoma to bleed after surgery. According to various embodiments, it may be desirable to provide a tracheostomy tube that enhances clotting.

According to various aspects of the disclosure, an endotracheal device may include an elongated body member having a first end and a second end, first and second lumens defined by the body member, and a funnel member proximal the second end. The first lumen may extend from a first opening in the first end to a first opening in the second end. The second lumen may extend from a second opening in the first end to at least one port in a wall of the body member proximal the second end. The funneling member may be configured to direct fluid toward at least one of the ports in the wall.

In some aspects of the disclosure, an endotracheal device may comprise an elongated body member defining at least one lumen. At least a portion of the elongated member may be configured to release a mucolytic agent.

In accordance with various aspects of the disclosure, a tracheostomy device may comprise an elongated body member defining a lumen, with the body member comprising a clotting agent.

### BRIEF DESCRIPTION OF THE DRAWINGS

FIG. 1 is a side, cross-sectional view of an endotracheal device in accordance with exemplary aspects of the disclosure;

FIG. 2 is an end, cross-sectional view along line II-II of FIG. 1;

FIG. 3 is a side, cross-sectional view of an endotracheal device in accordance with exemplary aspects of the disclosure;

FIG. 4 is a side, cross-sectional view of an endotracheal device in accordance with exemplary aspects of the disclosure; and

FIG. 5 is a side, partial cutaway view of a tracheostomy device in accordance with exemplary aspects of the disclosure.

### DETAILED DESCRIPTION

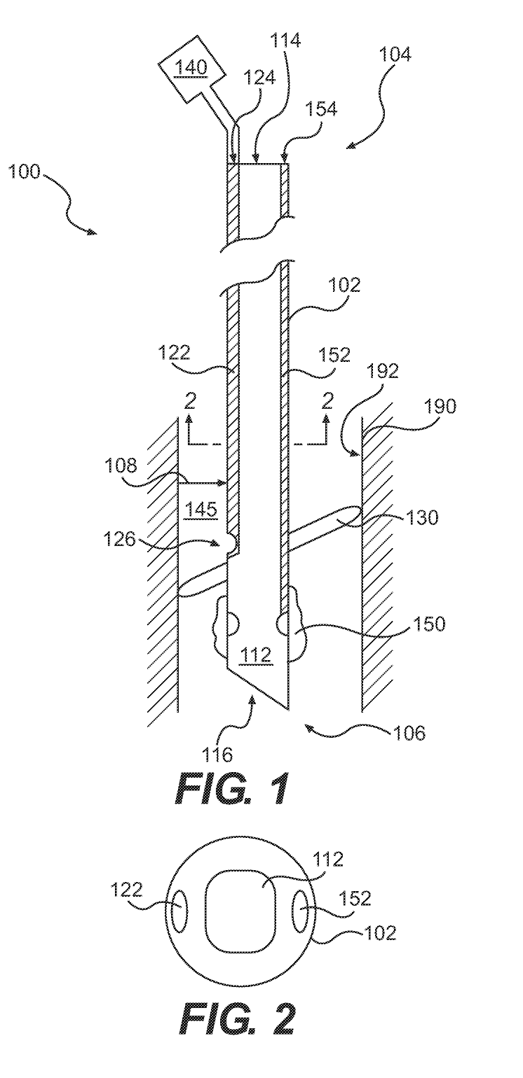

An exemplary embodiment of an endotracheal device 100 is illustrated in FIGS. 1 and 2. The endotracheal device 100 may comprise an elongated body member 102, for example, a flexible body member, having a first end 104 and a second end 106. The endotracheal device may be constructed from any suitable biocompatible material. According to various aspects, the elongated body member 102 may comprise a material that minimizes irritation to delicate mucosal tissue of the trachea and allows confirmation of tube placement due to condensation of the expired gases. For example, the body member 102 may be made from a non-toxic, thermo-sensitive, siliconized polyvinyl chloride (PVC). In some aspects, the body member 100 may be transparent or translucent. The body member 102 may have any size suitable compatible with a patient's anatomy. For example, the body member 102 may range in size from 3.0 to 20 mm, for example 5 to 10.5 mm in internal diameter (ID). The size may be chosen based on a patients body size, with the smaller sizes being used for pediatric and neonatal patients.

The elongated body member 102 may define a first lumen 112 extending from a first opening 114 in the first end 104 to a first opening 116 in the second end 106. The body member 102 may define a second lumen 122 extending from a second opening 124 in the first end 104 to at least one port 126 in a wall 108 of the body member 102 proximal the second end 106.

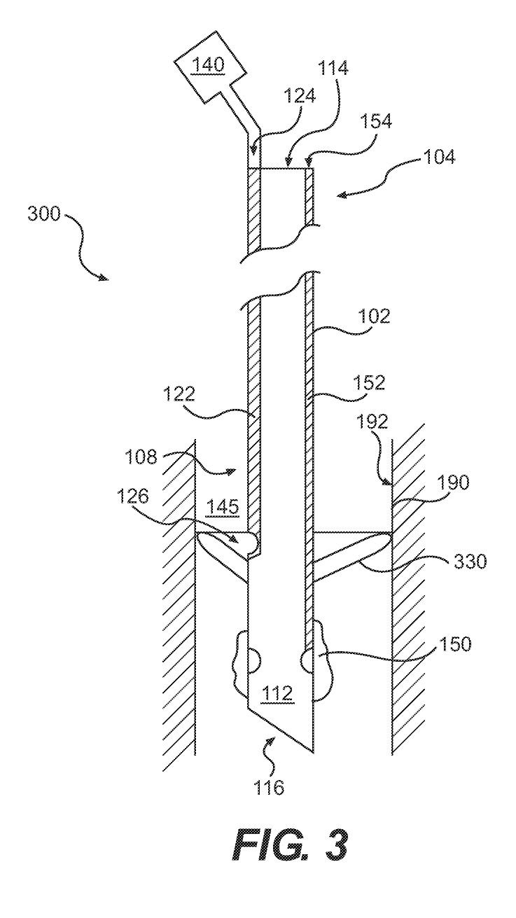

According to certain embodiments, the endotracheal device 100 may include a funneling member 130 proximal the second end 106 of the elongated body member 102. According to various aspects, the funneling member 130 may comprise a disc-shaped member. As shown in FIG. 3, according to some aspects, an endotracheal device 300 may include a funneling member 330 that comprises a frusto-conical member. The funneling member 130, 330 may be configured to direct fluid, for example, secretions, toward at least one of the ports 126 in the wall 108 of the body member 102.

According to various aspects, the funneling member 130, 330 may comprise a thin material constructed from, for example, silicone. The funneling member 130, 330 may be configured to cooperate with an inner wall 192 of a patient's trachea 190 to substantially form a tracheal seal to prevent aspiration of secretions.

According to various aspects, the endotracheal device 100, 300 may include a suction device 140 fluidly coupled to the second opening 124 in the first end 104 of the body member 102. The suction device 140 may be configured to aspirate fluids from a region 145 external the body member 102 proximal the part(s) 126 in the wall 108.

In some aspects, the endotracheal device 100, 300 may comprise an inflatable member 150 about the body member 102 proximal the second end 106. The elongated body member 102 may define a third lumen 152 (FIG. 2) extending from a third opening 154 in the first end 104 of the body member 102 to a portion of the body member 102 surrounded by the inflatable member 150. An air source (not shown) may be in fluid communication with the third lumen 152 via the third opening 154.

Once the endotracheal device 100, 200 is positioned in a patient's trachea 190, the air source may be operated to inflate the inflatable member 150 so that the inflatable member 150 may cooperate with the tracheal wall 192 to seal the lower airways (not shown) against air leakage and aspiration of secretions. The inflatable member 150 may be nearer the second end 106 relative to the port(s) 126 in the wall 108, such that secretions are maintained above the inflatable member 150 (when inflated) to facilitate suctioning thereof.

In accordance with various aspects, at least a portion of the endotracheal device 100, 300 may be configured to release at least one mucolytic agent. For example, in some aspects, a portion of the endotracheal device 100, 300 may be impregnated with the mucolytic agent(s). According to various aspects, the impregnated portion may include the funneling member 130, 330. In some aspects, a portion of the endotracheal device 100, 300 may comprise a coating containing the mucolytic agent(s). According to various aspects, the coated portion may include the funneling member 130, 330.

The mucolytic agent(s) may be chosen from, for example, acetyl cysteine, guaifenesin, and sodium thioglycolate. The mucolytic agent may prevent mucus from accumulating on the body member 102 as it is being removed and re-inserted. Some mucolytic agents such as, for example, acetyl cysteine, dissolve mucus. Some mucolytic agents such as, for example, guaifenesin, may function as expectorants.

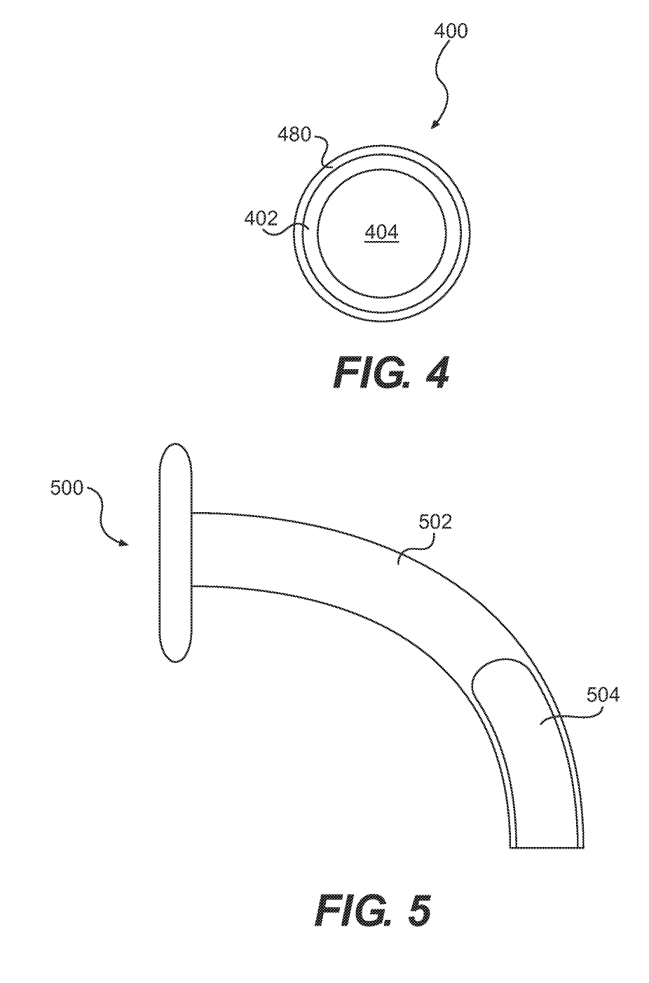

Referring now to FIG. 4, in accordance with various aspects of the disclosure, an endotracheal device 400 may comprise an elongated body member 402 defining a lumen 404. At least a portion of the endotracheal device 400 may be coated with at least one mucolytic agent 480. According to various aspects, a portion of the elongated body member may be impregnated with and configured to release at least one mucolytic agent For example, in some aspects, the mucolytic agent(s) 480 may be chosen from, for example, acetyl cysteine, guaifenesin, and sodium thioglycolate.

Sodium thioglycolate possesses free sulfhydryl groups that are expected to dissolve mucus by breaking disulfide bonds between gel-forming mucins. A 0.01-0.5% solution, for example, a 0.1% solution, of sodium thioglycolate in saline can dissolve human mucus.

According to some aspects, at least a portion of the endotracheal device 100, 300, 400 may be coated and/or impregnated with an antimicrobial agent. The antimicrobial agent may comprise, for example, a composition including silver. Some examples of antimicrobial agents consistent with this disclosure are described in U.S. Pat. No. 6,949,598, the disclosure of which is incorporated herein by reference in its entirety.

Referring now to FIG. 5, a tracheostomy device 500 may comprise an elongated body member 502 defining a lumen 504. According to various aspects, the body member 502 may comprise a clotting agent such as, for example, collagen. In some aspects, the body member 502 may be coated with the clotting agent. The clotting agent may aid in reducing localized (e g., stomal site) bleeding that may occur after a percutaneous tracheostomy.

According to another embodiment of the present disclosure, at least a portion of a drain tube, such as a chest drain, is coated and/or impregnated with a proteolytic agent. Suitable non-limiting examples of proteolytic agents include acetyl cysteine, guafenesin, and sodium thioglycolate. Examples of suitable drain tubes include those described in PCT/US07/60156, filed Jan. 5, 2007, the disclosure of which is incorporated herein by reference in its entirety.

### Example

Endotracheal tubes extubated from human patients were covered in a think layer of mucus Some tubes were washed in a 0.1% solution of sodium thioglycolate in saline, while a control group of tubes were washed in saline. The mucus was immediately dissolved from the tubes that were washed in thioglycolate, while the mucus layer remained intact in the saline control group tubes.

It will be apparent to those skilled in the art that various modifications and variations can be made in the medical devices and methods of the present invention without departing from the scope of the invention. Other embodiments of the invention will be apparent to those skilled in the art from consideration of the specification and practice of the invention disclosed herein. It is intended that the specification and examples be considered as exemplary only.

What is claimed is:

1. An endotracheal device, comprising:
an elongated body member having a first end and a second end;
a first lumen defined by the body member, the first lumen extending from a first opening in the first end to a first opening in the second end;
a second lumen defined by the body member, the second lumen extending from a second opening in the first end to at least one port in a wall of the body member proximal the second end; and
a funneling member proximal said second end, said funneling member being configured to direct fluid toward at least one of said at least one port in the wall.

2. The device of claim 1, wherein the funneling member comprises a disc-shaped member.

3. The device of claim 1, wherein the funneling member comprises a frustoconical member.

4. The device of claim 1, further comprising a suction device fluidly coupled to the second opening in the first end of the body member.

5. The device of claim 4, wherein said suction device is configured to aspirate fluids from a region external the body member proximal said at least one port in the wall.

6. The device of claim 1, further comprising an inflatable member about the body member proximal said second end.

7. The device of claim 1, further comprising a third lumen extending from a third opening in said first end of the body member to said inflatable member.

8. The device of claim 1, wherein at least a portion of said endotracheal device is configured to release a mucolytic agent.

9. The device of claim 8, wherein said portion of said endotracheal device is impregnated with said mucolytic agent.

10. The device of claim 8, wherein said portion comprises a coating containing said mucolytic agent.

11. The device of claim 8, wherein said mucolytic agent comprises at least one of acetyl cysteine, guaifenesin, and sodium thioglycolate.

12. The device of claim 1, wherein at least a portion of said endotracheal device is at least one of coated and impregnated with an antimicrobial agent.

13. The device of claim 12, wherein said antimicrobial agent comprises silver.

14. The device of claim 1, wherein said funneling member is configured to cooperate with an inside surface of a trachea to substantially form a tracheal seal.

15. An endotracheal device, comprising:
an elongated body member defining at least one lumen, at least a portion of said elongated member being configured to release a mucolytic agent.

16. The device of claim 15, wherein said portion of said device is impregnated with said mucolytic agent.

17. The device of claim 15, wherein said portion of said device is coated with a coating containing said mucolytic agent.

18. The device of claim 15, wherein said mucolytic agent comprises one of acetyl cysteine, guaifenesin, and sodium thioglycolate.

19. A tracheostomy device, comprising:
an elongated body member defining a lumen, said body member comprises a clotting agent.

20. The device of claim 19, said clotting agent comprising collagen.
